(19) United States
(12) Patent Application Publication
(10) Pub. No.: US 2001/0010906 A1
(43) Pub. Date: Aug. 2, 2001

(54) METHOD AND KIT FOR DIAGNOSING AND/OR QUANTIFYING BY SANDWICH HYBRIDISATION OF NUCLEIC ACID SEQUENCES ON SOLID SUPPORT

(76) Inventors: ISABELLE ALEXANDRE, LEXVE (BE); NATHALIE ZAMMATTEO, VERVIERS (BE); ISABELLE ERNEST, POUCHET (BE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/254,784

(22) PCT Filed: Sep. 9, 1997
(86) PCT No.: PCT/BE97/00102

§ 371 Date: None

(30) Foreign Application Priority Data
Sep. 9, 1996 (BE) 09600755

(51) Int. Cl. C12Q 1/68; C08F 120/56; C08F 220/56; C08F 8/40; C07H 21/04; C07H 21/02

(52) U.S. Cl. 435/6; 525/329.4; 525/340; 536/23.1; 536/25.4

(57) ABSTRACT

The invention concerns a method for detecting and/or quantifying a target nucleotide sequence (2) present in a biological sample characterised in that it comprises a “sandwhich” type contacting of the target nucleotide sequence (2) with a trapper single strand nucleotide sequence (5) fixed on an insoluble solid support (3) and complementary of part (7) of said target nucleotide sequence (2) and with one or several nucleotide sequences (6,11) of which at least one is marked (6), the said nucleotide sequence(s) (6,11) being complementary of another part (8) of the target nucleotide sequence (2); in that the trapper nucleotide sequence (5) is fixed covalently by one of its ends on the solid support (3), and has a length between 50 and 500 bases; and in that the part (10) of the trapper nucleotide sequence (5) which is not hybridised with the part (7) of the target nucleotide sequence (2) has less than 60 bases. The invention also concerns the kit for detecting and/or quantifying for implementing this method and the use of one standard quantifying nucleotide sequence.

### SUBJECT OF THE INVENTION

The present invention relates to a method and kit comprising reagents for the detection and/or quantification by sandwich-type hybridization of nucleic acid sequences on a solid support.

### TECHNOLOGICAL BACKGROUND FORMING THE BASIS OF THE INVENTION

It is known to covalently attach DNA to a support and to use it as a capture nucleotide sequence to attach a target or standard nucleotide sequence (intended for the quantification of a target sequence) and to be able to detect it directly, if it is attached to a detectable chemical molecule. In the case where the target or standard nucleotide sequence to be identified is not labeled, it is possible to carry out a “sandwich”-type hybridization using a labeled nucleotide sequence.

The labeled nucleotide sequence will be immobilized if the target or standard nucleotide sequence is present and immobilized on a trapping nucleotide sequence. The target or standard nucleotide sequence is then sandwiched between the trapping nucleotide sequence and the labeled nucleotide sequence, through a double recognition, which increases the specificity, reduces the background noise and allows quantification of the nucleotide sequences. This is only possible if the hybridization is carried out in a quantitative and reproducible manner. This is all the more true if the yield of this hybridization is high.

The importance of such a quantitative hybridization is the measurement of nucleic acids obtained from “biological agents” which are pathogenic or otherwise, such as viruses, fungi, bacteria, mycoplasmas, animal and plant cells or tissues, which are often amplified by an amplification step, such as PCR (Polymerase Chain Reaction (U.S. Pat. No. 4,965,188), LCR (Landegren et al., 1988, Science, 241, 1077-1080), NASBA (Kievits et al., 1991, J. Virol. Methods 35. 273-286), CPR (Cycling Probe Reaction (WO Patent 95/14106) or ICR.

However, the detection of the amplified sequences (called hereinafter amplicons) requires carrying out a specific detection which is sensitive and easily adaptable to a large number of samples.

A first solution consists in using beads, as described in Patent Application EP-0 205 532, where Sephacryl beads of 5 to 50 μm are activated by diazotization of aromatic amines. Moreover, multiwell plates already serve as a base for many tests, especially ELISA and many types of apparatus with photometry, fluorescence or luminescence reading already exist for reading these plates. However, it is necessary to be able to immobilize the target or standard nucleotide sequences on these plates in order to be able to detect them and/or quantify them.

This attachment may be obtained by simple adsorption (non-covalent reaction), and makes it possible to measure amplicons present in solution (Dahlem et al. 1987, Mol. Cell. Probes 1. 159-168; Cross et al. 1992, The Lancet 340. 870-873; Allibert et al., EP 486661). However, these methods suffer from a lack of control of the adsorption process (which requires work with very large fragments, often whole plasmids, and leads to the use of double-stranded trapping agents which can recombine) and from the difficulty of optimizing the size and the trapping nucleotide sequence because of the uncertainties linked to the adsorption.

In various state of the art documents, attempts have been made to optimize the length of a single-stranded sequence which can serve as trapping agent for a target sequence and to characterize the length of the target sequence necessary in order to be detected under the best conditions.

In the document “J. of Clin. Microbiol” (Vol. 28, June 1990, pp. 1469-1472), Inouye and Hondo et al. describe a method of direct hybridization on sequences attached to microplates of DNA segments of different lengths. This hybridization is carried out on DNA trapping segments noncovalently adsorbed on these microplates. Trapping agents of different lengths are adsorbed and hybridized with a biotinylated sequence of 642 base pairs. The same hybridization curve, but with an increasing hybridization efficiency as a function of the size of the fragments, is observed with the different sequences. On the basis of this experiment, the authors concluded that it is preferable that the target sequence to be identified is more than 300 base pairs in length. Since the adsorption of the DNA on the plastic occurs over very long lengths and occurs at several sites, this makes it impossible to know the lengths available for obtaining an efficient hybridization.

In the document “Analytical Biochem.” (No. 177, pp. 27-32 (1989)), Keller et al. describe a sandwich-type hybridization method using as trapping sequence a fragment of 3300 base pairs which serve for the detection of a target sequence of 190 base pairs which is also complementary to another labeled sequence, the single-stranded trapping sequence being covalently attached by an NH₂ functional group to a solid support. As appears in FIG. 2 of this document, the target sequence only very partially overlaps with the trapping sequence and the labeled sequence.

In the publication “Clin. Chem.” (No. 31, pp. 1438-1443 (1985)), Polsky-Cinky et al. describe a method in which the trapping sequence contains 4800 base pairs of which 800 base pairs are complementary to the target sequence of 1600 base pairs to be detected. As appears in FIG. 1 of this document, the target sequence is complementary in the hybridization of another labeled nucleotide sequence.

Japanese Patent Application JP-8089300 describes a trapping sequence having a length of less than 30000 base pairs and containing at least 10 repeating units of 100 base pairs capable of hybridizing in series in the same orientation with target sequences to be detected and increases the sensitivity of the traditional methods.

Patent Application EP-0 079 139 describes a single-stranded trapping sequence of 1200 to 1500 base pairs hybridizing with target sequences of 600 to 700 base pairs, which are detected by a sandwich hybridization by means of a labeled sequence complementary to another portion of the target sequence.

However, in all these hybridization devices based on a simple hybridization or a sandwich hybridization, poor overlapping is observed between the trapping sequence and the target sequence, and a poor overlapping is observed between the target sequence and the trapping sequence and/or the labeled sequence. Consequently, by using very long trapping sequences for the detection or very long target sequences to be detected, there is folding of the sequences over themselves or with other “interfering” sequences present in the sample (sequences complementary to an amplicon sequence, and the like).

In the document “Nucleic Acid Research” (Vol. 21, No. 15, pp. 3469-3472 (1993)), Kosaka et al. describe trapping sequences attached by a covalent bond to microplates. These very short trapping sequences (of the order of 17 base pairs) are used to obtain hybridization of target sequences to be identified or to be quantified in the presence of similar labeled sequences. As appears in FIG. 2 of this document, in spite of the short length of the trapping sequence, there is no optimum overlap between this trapping sequence and the target sequence to be quantified.

In the document “Clin. Chem.” (No. 40/2, pp. 200-205 (1994)), Rasmussen et al. describe a single-stranded trapping sequence attached by a covalent bond to microplates. This very short trapping sequence (25 nucleotides) is used to obtain the hybridization of target sequences of 350 to 500 base pairs.

In Patent Application WO94/06933, there is described a method of sandwich hybridization by means of a trapping sequence attached to microplates by sandwich hybridization of a target sequence hybridizing with a labeled sequence. However, in the exemplary embodiments, the trapping sequences consist of about 20 nucleotides used for the hybridization of target sequences of 400 to 600 base pairs. When the trapping nucleotide sequence is very short, poor overlapping of the target sequence and folding of the target sequence over itself or rehybridization of target sequences with themselves are observed. Furthermore, the low percentage of hybridization of the target sequence with the trapping sequence reduces sensitivity.

In the document “Journ. of Clin. Microbiol.” (Vol. 33, pp. 752-754 (1995)), Shaw et al. describe a method using a single-stranded trapping sequence of 188 base pairs adsorbed on microplates, this biotinylated trapping sequence making it possible to hybridize a target biotinylated sequence of 245 base pairs. This hybridization technique which is not based on a sandwich-type recognition involves poor overlapping between the trapping sequence and the target sequence, and in all cases, poor overlapping of the target sequence over at least approximately 60 base pairs. Consequently, this portion of 60 base pairs is sufficient to obtain folding of the target sequence, or even of the trapping sequence, over itself, in which the secondary structures are capable of disrupting the hybridization between the trapping sequence and the target sequence, which reduces the sensitivity of the test.

In the document “Journ. of Chem. Microbiol.” (Vol. 9, pp. 638-641 (1991)), Keller et al. describe a single-stranded trapping sequence attached (in a random manner through the use of diaminohexane) by a covalent bond to microplates, this single-stranded trapping sequence being 126 base pairs in length, of which at least a portion is capable of hybridizing with a target sequence of 191 base pairs. The document mentions that the single-stranded sequence is complementary to only a portion of the target sequence of 191 base pairs, but is not capable of obtaining a complete overlap, in particular with the primer sequences on either side of this target sequence so as to avoid any homology in overlapping between the primers and the single-stranded sequence. More than 30 base pairs are therefore observed at the two ends of the target sequence which do not overlap with the trapping sequence, recognition of the hybridization being detected by labeling the target sequence with attached biotin and may be recognised by the streptavidin-peroxidase conjugate allowing its detection by colorimetry. Poor overlapping of the target sequence to be quantified and folding of the target sequence and of the end of the trapping sequence attached to the solid support are obtained. Furthermore, the attachment of the trapping agent to well occurs in a random manner and the portion of the trapping sequence accessible to the target sequence is not known.

Patent Application EP 0 205 532 describes a trapping sequence of 341 base pairs covalently attached to microbeads, this trapping sequence being capable of reacting with a target sequence in order to obtain an overlap of 175 base pairs between the trapping sequence and the target sequence, the target sequence reacting with a labeled sequence of 201 base pairs by sandwich hybridization. However, this document does not describe an optimum overlap between the single-stranded trapping sequence and the target sequence. A free portion of 166 base pairs is observed at the level of the single-stranded trapping sequence in which folding of the single-stranded trapping sequence over itself or the pairing with other sequences present in the sample are capable of reducing the percentage of target sequence hybridizing with the trapping sequence, and the sensitivity of the detection and/or quantification.

Gene amplification methods (PCR, LCR, NASBA) for the purpose of screening and adding nucleic acids also has problems at the level of an optimum quantification of said nucleic acids. Indeed, in a quantification step, a detection and a quantification must be carried out in each of the 3 steps of the assay, namely:

1) the quantitative extraction (often complex) of the nucleic acid in the biological sample;

2) the quantitative amplification of the sequence studied; and

3) the quantitative measurement of the number of amplified sequences (called amplicons).

These methods of extraction have improved over the past few years and the yield of these extractions is often greater than 90%, and makes it possible to consider this step as nonlimiting in the entire process of quantifying the nucleic acid.

The amplification step, especially by the Polymerase Chain Reaction (PCR) (U.S. Pat. No. 4,965,188), poses great difficulties as regards the control of the quantification and of the detection.

In the amplification, the first step consists in unpairing the DNA double strands, which are often very long (and optionally stabilized by various proteins or molecules), and in increasing the temperature so that the two strands are separated.

The second step is the annealing of the primer oligonucleotides. These are in great excess relative to the DNA to be amplified and conditions can be found in which this recognition is optimal. Next comes the extension of the DNA using primers by DNA polymerase, which should occur under optimum conditions (pH, temperature, salts, dNTP, and the like) in order to encounter the Primer-DNA attachment sites. Even under optimum conditions, it is observed that the yield (or level) of amplification, that is to say the average proportion of molecules which duplicate during one cycle does not exceed 90% and may even be much less than this value (J. Peccoud, 1993, Med/sci, 9, 1379).

Furthermore, there is variability from one sample to another, for the same sample depending on the dilution and even from one tube to another for the same sample (J. Peccoud, 1993, Med/sci, 12, 1378-1385).

The method of amplification by Ligase Chain Reaction (LCR) (Landegren et al., 1988, Science, 241, 1077-1080) and by NASBA exhibit the same difficulties in estimating the level of amplification and therefore the quantification of the target sequences to be measured.

There are several means of obtaining quantification of the target nucleotide sequence. Since the amplification depends on a large number of factors and variations are observed, a standard which is as close as possible to the target sequence and if possible which is amplified in the same tube must be used as reference. An internal standard (that is to say placed in the same PCR tube and amplified at the same time as the target) is preferred to comparison with an external standard which would be amplified in parallel with the test. The use of an external standard is only possible in the case where the method of amplification is standardized and reproducible.

A constraint exists however which is that of the quantitative and separate detection of the 2 amplicons. For the efficiency of the amplification to be the same, the 2 sequences should be as similar as possible, while retaining the possibility of being able to differentiate them during their determination. Furthermore, if the efficiency is kept constant during a whole series of cycles, a slowing down of this efficiency is observed at the end of the amplification and finally becomes zero. A plateau effect is obtained for which the number of amplicons no longer increases with the number of cycles. This slowing down appears at different points of the PCR depending on the number of copies present at the beginning. This complicates the use of the internal standard which continues to be amplified when the target has already reached the plateau. If the difference in concentration between the 2 sequences is too large, one of the 2 will reach a plateau while the other will still not be in sufficient concentrations to be measured. These constraints often lead the authors to limit themselves to internal standard concentrations which are very similar to those of the target sequences and to work in the logarithmic amplification zone of the PCR, that is to say with a reduced number of amplicons.

Patent Application WO96/09407 describes a method of amplification comprising the use of an internal standard having a specific portion different from the target nucleotide sequence to be quantified by 17 amino acids. In this case, the target and standard sequences of the same length are quantified by attaching them to biotins which react with a straptavidin attached to a solid support.

Patent Application WO93/10257 describes a method of quantifying a DNA fragment by adding an internal standard which is different from the target DNA fragment to be quantified by less than 10% in terms of sequence and/or size. The standard nucleotide sequence differs from the target DNA fragment by a specific sequence containing a deletion, mutation or addition at a site of 1 to 5 nucleotides allowing the incorporation of a restriction or cleavage site, which can be achieved by an enzyme or any other means. The quantification is carried out by specific recognition of the target DNA fragment or of the standard nucleotide sequence by different specific primers. The use of different primers which hybridize with the fragments in different sites will generate labeled fragments of different sizes and sequences, which can be separated by electrophoresis. This method is based on a double verification of the specificity of identification. However, such methods and devices based on the selective identification of the standard sequence in one step do not guarantee sufficient specificity and sensitivity, which can lead to the presence of false-positives or false-negatives during quantification of a target nucleotide sequence.

### AIMS OF THE INVENTION

The present invention aims to provide a new method and a kit allowing detection and/or quantification of nucleic acid sequences which do not have the disadvantages of the state of the art cited.

A specific aim of the present invention is to provide a method and a kit allowing optimum hybridization of the nucleic acid sequences, in particular a high percentage of hybridization of the trapping sequence with a target or standard sequence, a low risk of folding of these sequences or of the trapping sequence over itself and a low risk of new pairing of these sequences via complementary sequences present in the sample.

Another aim of the present invention is to provide a detection and/or quantifying method and kit exhibiting improved specificity and sensitivity compared with the methods and devices of the state of the art, in particular for the detection and/or quantification of any type of nucleic acids present in a biological sample and optionally obtained after gene amplification.

An additional aim of the present invention is to obtain a method and kit for detecting and/or quantifying said target nucleic acid sequence which allows the amplification of an internal or external standard sequence regardless of the number of gene amplification cycles.

### CHARACTERISTIC FEATURES OF THE INVENTION

The present invention relates to a method for detecting and/or quantifying a nucleotide sequence called “target” present in a biological sample, characterized in that it comprises a bringing into contact of the “sandwich” type of said target nucleotide sequence 2 with a nucleotide sequence called “trapping sequence” 5 attached to an insoluble solid support 3, said trapping nucleotide sequence being complementary to a portion 7 of the target nucleotide sequence, the bringing into contact of the “sandwich” type being also carried out with one or more other nucleotide sequences (6, 11) of which at least one (6) is labeled, said nucleotide sequence(s) (6, 11) (of which at least one (6) is labeled) being complementary to another portion 8 of the target nucleotide sequence 2 (another portion different from that 7 hybridized via the “trapping” nucleotide sequence 5); in that the trapping nucleotide sequence 5 is covalently attached by one of its ends to the solid support 3; in that the trapping nucleotide sequence 5 has a length of between 50 and 500 bases, preferably between 100 and 300 bases, more particularly between 120 and 250 bases; and in that a portion 10 of the trapping nucleotide sequence 5 which does not hybridize with the portion 7 of the target nucleotide sequence 2 is less than 60 bases, preferably less than 40 bases or less than 20 bases, or even zero.

According to a preferred embodiment of the invention, the portions 13 of the target nucleotide sequence 2 which do not hybridize with the trapping nucleotide sequence 5 and with the nucleotide sequence(s) (6, 11) (of which at least one (6) is labeled), is less than 60 bases, preferably less than 40 bases, or even zero.

In the remainder of the description, the nucleotide sequence(s) (6, 11) (of which at least one (6) is labeled) will be called “helper” nucleotide sequences 11 when said sequence(s) are not labeled, and “labeled” nucleotide sequences 6 when they are capable of being recognised directly or indirectly by a detection and/or quantification system, preferably chosen from the group consisting of fluorescence, chemiluminescence, electroluminescence, staining, detection by radioactive labeling, bioluminescence, electrochemistry, light reflection, an optical method or a mixture thereof.

The “helper” nucleotide sequences 11 are used to stabilize the “sandwich” and to obtain an overlapping which is as complete as possible of the target nucleotide sequence 2 with the labeled nucleotide sequence 6 and with the trapping nucleotide sequence nucleotide sequence 5, which increases the sensitivity and the specificity of the method according to the invention.

In the detection and/or quantification method, the sandwich hybridization is preferably carried out in two steps, that is to say that the first step consists of the hybridization of the target nucleotide sequence 2 with the trapping nucleotide sequence 5 and that the second step is the hybridization of the target nucleotide sequence 2 with one or more nucleotide sequences (6, 11) of which at least one (6) is labeled. The two steps are preferably separated by a washing step. In the preferred embodiment, the sequences are chosen so that the conditions (temperature, concentration of salt, reaction time) are compatible for both hybridizations, which makes it possible to carry out the hybridization in a single step.

According to the invention, the target and standard nucleotide sequences to be detected and/or quantified consist of any type of nucleic acid, DNA or RNA. Preferably, the trapping, “helper”, labeled and standard nucleotide sequences used according to the present invention consist of DNA so as to avoid any destruction of these sequences by RNases which may be present in the biological sample.

Advantageously, the target 2 and standard 1 nucleotide sequences result from a preliminary amplification by a gene amplification method, preferably chosen among the group consisting of PCR, LCR, CPR, NASBA or ICR.

According to the invention, in the case where the target nucleotide sequence 2 is an amplicon resulting from a gene amplification, the 5′ terminal portion 9 of the target nucleotide sequence 2 may be left nonoverlapped by the labeled sequence(s) 6 and the “helper” nucleotide sequence(s) 11, and is then over-lapped by a complementary “primer” nucleotide sequence 12 used for the amplification of the target nucleotide sequence 2. According to the invention, this “primer” nucleotide sequence 12 may be considered to be a “helper” type sequence. It is therefore possible to also obtain optimum, or even complete, overlapping of the target nucleotide sequence 2 with the trapping nucleotide sequence 5, the labeled nucleotide sequence(s) 6 and possibly the “helper” nucleotide sequences 11 and the primer 12.

The invention also relates to a method for quantifying a target nucleotide sequence 2 present in a biological sample, which comprises the following steps:

a preparation of a known quantity of a standard nucleotide sequence 1 possessing at least a portion A common to the target nucleotide sequence 2 and a specific portion B whose sequence is different and possesses a content of GC/AT bases similar, preferably identical, to the sequence of a specific portion B of the target nucleotide sequence 2,

an optional extraction of the target nucleotide sequence 2 to be quantified from the biological sample,

an optional amplification of the target nucleotide sequence 2, and

a bringing into contact of the “sandwich” type of the target 2 and standard 1 nucleotide sequences with a trapping nucleotide sequence 5, preferably as described above, the trapping nucleotide sequence 5 being complementary to the common portion of the target nucleotide sequence and the standard nucleotide sequence, the bringing into contact of the “sandwich” type being also carried out with one or more nucleotide sequences (6, 11) of which at least one (6) is labeled and complementary to the specific portion B of the target nucleotide sequence 2 or the specific portion B of the standard nucleotide sequence 1,

said method also comprising a quantification of the ratio between the specific labeling of the target nucleotide sequence 2 and the specific labeling of the standard nucleotide sequence 1. “Content of GC/AT bases similar” is understood to mean that the ratio of GC/AT bases of the standard is different by less than 20% from the ratio of GC/AT bases of the target nucleotide sequence.

Conseqently in the method of quantification according to the invention, the bringing into contact of the “sandwich” type as described above is advantageously combined or otherwise with a device for quantification using an internal or external standard nucleotide sequence.

The specific portions B of the standard and target nucleotide sequences preferably correspond to the portion 8 of the target or standard nucleotide sequences. The common portion A preferably corresponds to the portion 7 of this target or standard nucleotide sequence which hybridizes with the trapping nucleotide sequence 5.

According to the invention, the specific portion B of the standard nucleotide sequence 1 is different from the specific portion B of the target nucleotide sequence by 5 to 500 nucleotides, preferably by 20 to 40 nucleotides.

Advantageously, the internal standard nucleotide sequence 1 contains on either side of the specific sequence B and the common sequence A portions 15 which are common to portions 15 of the target nucleotide sequence 2, and capable of serving in whole or in part as sequences complementary to primer sequences 12 for gene amplification.

The method according to the invention is particularly suitable for the use of internal standard nucleotide sequences conjointly amplified with the target nucleotide sequence 2 or of external standard amplified in parallel with the target nucleotide sequences 2.

Advantageously, the method according to the invention comprises a large number of gene amplification cycles, preferably by PCR or LCR, preferably greater than 30.

According to a preferred embodiment of the invention, the internal standard is added in a variable quantity to the initial sample and the ratio obtained between the specific labeling of the target nucleotide sequence 2 and the specific labeling of the standard nucleotide sequence 1 is plotted on a graph as a function of known quantities added to the initial sample, making it possible to determine on the straight line thus obtained, for a ratio equal to 1, the quantity of target nucleotide sequence 2 present in the sample.

According to another preferred embodiment of the present invention, the standard nucleotide sequence is added in an identical quantity to a sample having undergone various dilutions, and the ratio between the specific labeling of the target nucleotide sequence and the specific labeling of the standard nucleotide sequence is plotted on a graph as a function of the dilutions of the sample, and the straight line obtained makes it possible to determine, for a ratio equal to 1, the quantity of target nucleotide sequence 2 present in the sample. The quantification may also be carried out by comparing the ratio obtained with a single determined quantity of standard added to a single quantity of sample and a calibration straight line.

The present invention also relates to the detection and/or quantification kit comprising the reagents for carrying out the methods described above.

The kit for detecting and/or quantifying, by a “sandwich”-type hybridization, a target nucleotide sequence 2 comprises a trapping nucleotide sequence 5 attached to an insoluble solid support 3 complementary to a portion 7 of the target nucleotide sequence 2 and one or more other nucleotide sequence(s) (6, 11) (of which at least one (6) is labeled), said nucleotide sequence(s) (6, 11) being complementary to another portion 8 of the target nucleotide sequence 2. In the detection and/or quantification kit according to the invention, said trapping nucleotide sequence 5 is covalently attached by one of its ends to the solid support 3 and has a length of between 50 and 500 bases, preferably between 100 and 300 bases, more particularly between 120 and 250 bases, and the portion 10 of the trapping nucleotide sequence 5 which does not hybridize with the portion 7 of the target nucleotide sequence 2 is less than 40 bases, preferably less than 20 bases, or even zero.

It is clearly understood that in the method and kit according to the invention, the labeled nucleotide sequence has a sufficient length to specifically recognise the target or standard nucleotide sequence to be detected and/or quantified, this specificity depending on the type of target or standard nucleotide sequence to be detected and/or quantified, and may be characterized by a recognition through hybridization of a specific portion complementary to at least 10 bases, preferably more than 20 bases, of a target or standard nucleotide sequence.

The kit according to the invention will also comprise the reagents necessary for the specific identification of the labeled sequence, for the detection and/or quantification by a method preferably chosen from the group consisting of fluorescence, chemiluminescence, electroluminescence, staining, detection by radioactive labeling, bioluminescence, electrochemistry, reflection of light, an optical method or a mixture thereof.

The trapping nucleotide sequence 5 is covalently attached to the insoluble solid support 3 and is preferably produced through a terminal 5′ phosphate of the trapping nucleotide sequence 5 on one or more amine functional groups of the insoluble solid support 3 by reaction with carbodiimide.

The insoluble solid support 3 is preferably chosen from the group consisting of tubes, filters, beads, which may be magnetic, multiwell plates, a plate or a mixture thereof.

The invention also relates to a detection and/or quantification kit which comprises an internal or external standard nucleotide sequence 1 as described below and all the components necessary for the extraction, amplification, detection and/or quantification according to the methods described above.

The present invention will be described in greater detail in the examples below with reference to the accompanying figures.

### BRIEF DESCRIPTION OF THE FIGURES

FIG. 1 represents a schematic example of sandwich hybridization according to the invention.

FIG. 2 represents a concentration curve for CMV target DNA obtained after sandwich hybridization and bioluminescence detection.

FIG. 3 represents a sensitivity curve for Chlamydia trachomatis DNA measured after amplification by PCR, sandwich hybridization and detected by colorimetry by the streptavidin-peroxidase conjugate.

FIG. 4 represents a diagram illustrating the nucleotide structure of a typical competitive standard, compared with that of a target DNA to be measured during an amplification by PCR.

FIG. 5 represents a diagram of a competitive standard (A) for the measurement of CMV viral DNA and its comparison with the target DNA (B). The numbering corresponds to the “immediate early gene” nucleotide sequence (Demmler et al., 1988, J. Infectious Diseases, 158, 1177-1184).

FIG. 6 represents a diagram of a competitive standard (A) for the measurement of HIV virus RNA and its comparison with the target RNA (B). The numbering corresponds to the nucleotide sequence of the viral RNA (“Los Alamos de HIV”, Meyers et al. (1992)).

FIG. 7 is a schematic description of the principle of the quantification of a target DNA using a competitive standard according to the invention and their respective measurement using specific probes after capture on an immobilized common sequence.

FIG. 8 represents a curve of the concentration of CMV target DNA and of the corresponding standard by sandwich hybridization and measured by spectrophotometry.

FIG. 9 represents a curve of concentration of CMV target DNA and of the corresponding standard after amplification by PCR and sandwich hybridization using a common trapping probe and a biotinylated probe specific for the target DNA or the standard.

FIG. 10 represents the measurements of the CMV target DNA and of the internal standard after an amplification by 40 PCR cycles when 1000 copies of standard were added to increasing quantities of target DNA and measured after PCR by sandwich hybridization as defined in FIG. 7.

FIG. 11 represents the calibration straight line for a CMV target DNA using an internal standard according to the invention. The x-axis represents the ratios of the target and standard signals obtained in FIG. 10.

FIG. 12 represents the quantification of a CMV target DNA as a function of the number of PCR cycles using a competitive standard according to the invention. A competitive standard for the CMV sequence as described in FIG. 5 was used and added in a constant quantity to the sample which has undergone 4 different dilutions.

FIG. 13 represents a competitive RT-PCR carried out on an HIV target RNA and a standard RNA (FIG. 6) and a bioluminescence detection after sandwich hybridization. The graph represents for each RT-PCR the ratio between the detection obtained using a probe specific for the target and that for the standard. The results show a competition between the amplification of the target added in a constant quantity (10⁸ copies) compared with the standard added in a decreasing quantity ranging from 10¹⁰ (1), 10⁸ (2) to 10⁶ (3) copies.

### DESCRIPTION OF A PREFERRED EMBODIMENT OF THE INVENTION

The trapping nucleotide sequence 5, preferably a DNA sequence, is covalently attached to multiwell plates 3. This covalent attachment is obtained through the binding of a phosphate situated at the 5′ terminal position of the trapping sequence onto an amine situated on the support in the presence of carbodiimide, as described by Rasmussen et al. (1991, Anal. Biochem. 198, 138-142).

The attached trapping nucleotide sequences 5 may hybridize a target DNA 2, which will then be sandwich hybridized with a labeled nucleotide sequence 6.

One advantage of the sandwich hybridization is a very low background noise which makes it possible to carry out a massive analysis of clinical samples with a minimum of DNA purification.

The quantity of trapping nucleotide sequences attached to the multiwells may be up to 1.2 pmol for small oligonucleotides, but as regards larger nucleotide sequences, the quantity attached is smaller (of the order of 300 fmol for nucleotide sequences of about 500 bases). The quantity of trapping nucleotide sequences attached is sufficient not to be limiting in the hybridization method of the invention.

The attachment of the trapping nucleotide sequence onto microbeads also makes it possible to increase the number of trapping nucleotide sequences in the reaction solution using a larger quantity of beads.

The trapping nucleotide sequences according to the invention which are chosen are complementary over their entire length to the target nucleotide sequence to be detected. This advantageously allows easy production of these trapping nucleotide sequences by PCR from target nucleotide sequences cloned into plasmids and can thus serve for a reproducible industrial production of these trapping nucleotide sequences.

It is however possible to use trapping agents in which a portion of the nucleotide sequence comprises (a) sequence portion(s) not complementary to the sequence of the target. The inventors have tested trapping nucleotide sequences possessing a sequence of 20 or 40 bases not complementary to the target nucleotide sequence and which is situated at the 5′ terminal position serving for the attachment onto the multiwells.

These sequences serve as “spacers” between the trapping sequence proper and the solid support. In this experiment, the sequence bearing a “spacer” of 20 bases is more efficient than spacers of 40 and 60 bases for hybridizing small target sequences. This result is probably due to the additional possibilities of folding of these trapping nucleotide sequences over themselves, which reduces, or even inhibits, the hybridization of the target nucleotide sequences. It has been possible to visualize these folds using programs for predicting secondary structures Oligo4 and DNA-fold and they may effectively involve sometimes fairly long portions of the trapping sequence.

These folds should be avoided as much as possible because they reduce the hybridization efficiency, especially when they involve portions not complementary to the target sequence which cannot therefore displace them.

Another property of the method and of the kit of the invention is the advantageous use of trapping nucleotide sequences having a minimum size of 50 bases, if possible 100 and at best 150 or more bases. This observation is unexpected on the basis of the following considerations.

The stability of the hybrids, in the case where the ionic strength is constant and the size sufficient (above 50 bases), now depends only on the composition (%G+C) and not on the size of the hybrids. The importance of the size for promoting the hybridization or otherwise depends on the speed of the hybridization and not on their stability. Effectively, in solution, the size of the strands of nucleic acids influences the speed of pairing again. The latter is proportional to the square root of the length (Wetmur, J. G. and Davidson, N. 1968, J. Mol. Biol. 3, 584) and in the case where the 2 strands are of a different size, the speed is proportional to the square root of the shortest strand, whether a DNA (Wetmur, J. G. 1971, Biopolymers 10, 601) or an RNA (Hutton, J. R. and Wetmur, J. G. 1973, J. Mol. Biol. 77, 495).

The other factor which influences the speed of hybridization is the respective concentration of the target nucleotide sequences and of the trapping nucleotide sequences. The situation in this case is complex because two reactions are possible: on the one hand, the target nucleotide sequences to be measured being usually double-stranded in the starting solution, they will be able to recombine with each other and, on the other hand, they will be able to hybridize with the trapping nucleotide sequence. Two competitive reactions are therefore involved, one occurring in solution (the recombination of the double strands of the target nucleotide sequence) and the other on a solid support (the hybridization onto the trapping nucleotide sequence).

It is possible to consider first of all the situation where the quantity of trapping nucleotide sequence is in excess relative to the quantity of target nucleotide sequence. Target nucleotide sequence concentrations of the order of the fmol (from 0.1 to 50 fmol) are detected whereas the quantity of trapping nucleotide sequence may be up to 300 fmol.

If the 2 competitive reactions occured in solution, the kinetic formula which expresses the reduction in the concentration of single-stranded target nucleotide sequence (DNA) in solution (Cs) as a function of time is:

$$- \frac{ⅆ ( Cs )}{ⅆ t} = {k}_{1} [ Cf ] [ Cs ] + {{k}_{2} [ Cs ]}^{2}$$

in which:

k₂: kinetic constant of reassociation of the DNA

k₁: kinetic constant of hybridization with the nucleotide sequence

Cf : concentration of the nucleotide sequence which hybridizes with the DNA.

Since the reaction is carried out on an insoluble solid support, it is necessary to take into account the speed of diffusion (J) of the single-stranded target nucleotide sequence towards the solid surface onto which the trapping nucleotide sequence is attached, that is to say:

$$- \frac{ⅆ ( Cs )}{ⅆ t} = J + {k}_{1} [ Cf ] [ Cs ] + {{k}_{2} [ Cs ]}^{2}$$

The situation becomes very complex and cannot be understood in its entirety. Anderson and Young (1985) have tried to measure the influence of the size of the double-stranded target nucleotide sequence on filter hybridization in the presence of an excess of bound nucleotide sequence and they conclude “The difference in dependence on molecular weight of the two types of filter hybridization is not understood”.

As regards the influence of the size of the trapping nucleotide sequence on a plastic solid support, the studies have not yet been carried out and it is not possible to obtain a mathematical expression which makes it possible to predict the results obtained with the method of the invention.

If the specific conditions of the reaction medium are examined nevertheless, it is observed that the quantity of trapping nucleotide sequence (for example 300 fmol) is greatly in excess relative to the quantity of target nucleotide sequence (for example 10 fmol). If it is considered that this excess makes it possible to compensate for the limitations due to diffusion, the situation is similar to the reaction in solution where the speed of hybridization ought to be proportional to the square root of the length of the sequences.

By choosing a trapping nucleotide sequence of 360 bases instead of 180 bases, a shift in the yield from 30% to 50% is observed, that is to say an increase of 1.67 fold. Even based on a size effect in solution, that is to say at a speed dependent on the square root of the length, a maximum increase of 1.4 fold should have been expected. In addition, a yield of 50% has never been described up until now in the scientific literature.

Comparing the hybridization yields as a function of small trapping nucleotide sequences ranging from 50, 100, 150 and 250 bases, an increase in yield of 4, 6 and 17 fold is observed (taking the hybridization on the trapping nucleotide sequence of 50 bases as reference) whereas the square roots of these sequences are in a ratio of 1.4, 1.7 and 2.2 fold respectively.

An unexpected effect of the length of these trapping nucleotide sequences on the increase in the yield of hybridization is therefore observed.

A specific application of these high yields of hybridization of the target DNA on an insoluble support is its use for purifying one of the two strands of this target DNA. Indeed, the trapping agent consists of a single strand because a single primer phosphorylated at the 5′ terminal position is used during its construction by PCR. This phosphate is the only one which is able to react with the amine-containing support. After attachment, the plate is washed in the presence of 0.4 N NaOH (cf. Example 1) in order to remove the second strand. Only one strand attached to the support therefore remains. This strand being complementary to only one of the two strands of a target DNA, it will bind this strand. After washing, this hybridized strand may be easily dehybridized, for example, by heating or with 0.4 N NaOH. A single strand of DNA is thus obtained in the solution. This technique can be used on a large scale, for example, using beads on which the trapping nucleotide sequences are attached. This single strand preparation can have many applications as reagents using a chemically labeled strand for example.

The detection of non-labeled target sequences is carried out using their hybridization to trapping nucleotide sequences attached onto an insoluble support using one or more nucleotide sequences of which at least one is labeled (detection nucleotide sequences) which can hybridize to the portion of the target nucleotide sequence not recognised by the trapping nucleotide sequence. This sequence may be chemically labeled and detected according to the various methods known to persons skilled in the art. It is possible to obtain an attachment of at least 80% of the detection nucleotide sequence relative to the target nucleotide sequence hybridized to the trapping nucleotide sequence.

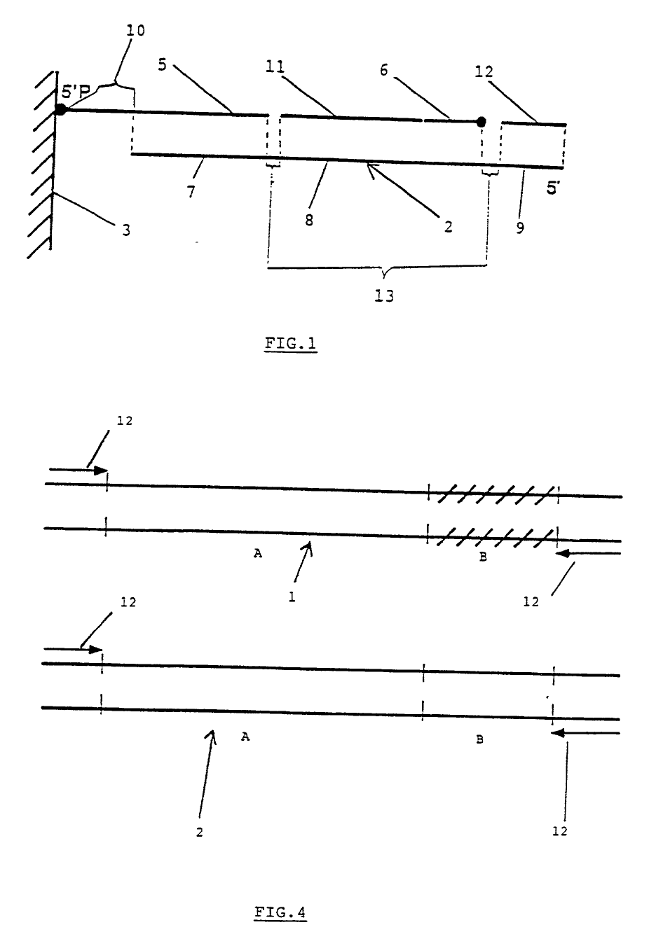

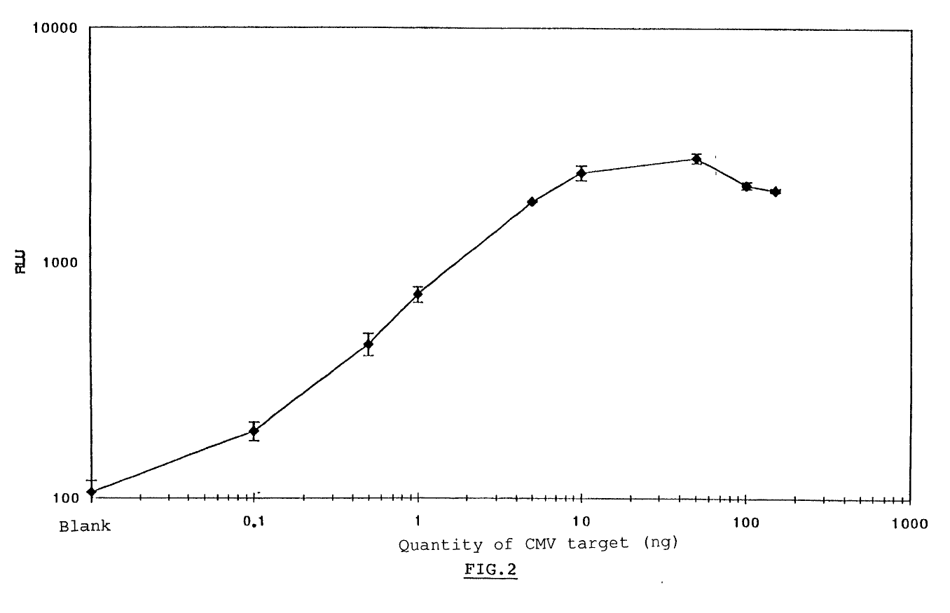

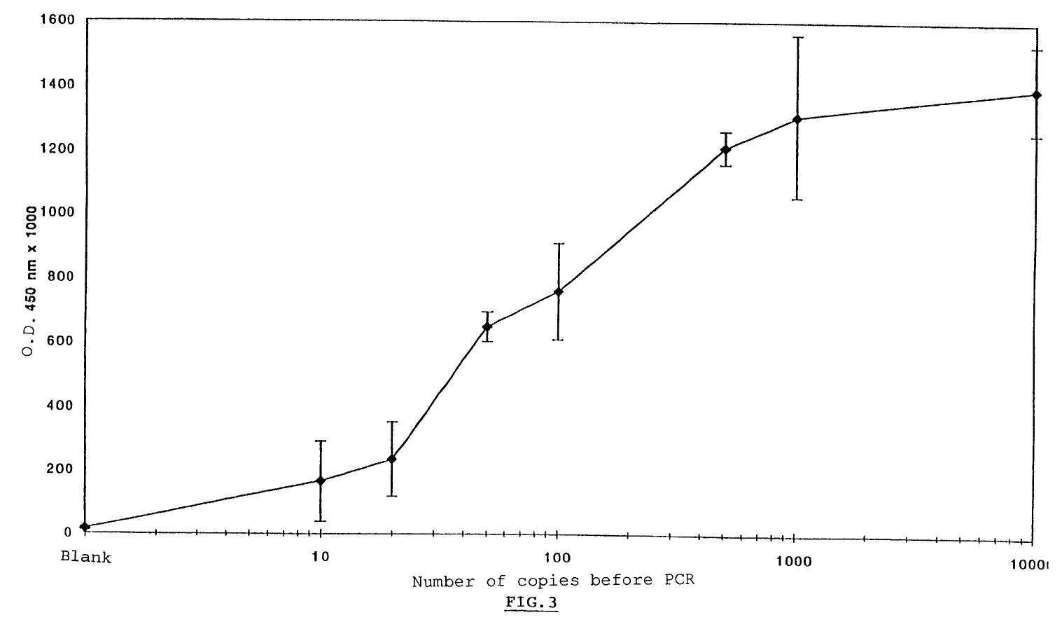

By examining the influence of the length of this labeled nucleotide sequence on the efficiency of the sandwich hybridization, it is possible to see the importance of using a labeled nucleotide sequence which overlaps as much as possible with the target nucleotide sequence. The best yields are obtained when the target nucleotide sequence overlaps completely, on the one hand, with the trapping nucleotide sequence and, on the other hand, with the detection nucleotide sequence (FIG. 1).

In the case of the detection of amplicons obtained by PCR, it is possible to leave on the 5′ terminal side of the target nucleotide sequence a sequence not overlapping with the detection nucleotide sequence but which can be recognised by the primers still present in the PCR solution. In this manner, the entire target nucleotide sequence is covered during the hybridization and there is no interference between the primers and the nucleotide sequence during the hybridization.

The increased efficiency obtained by this procedure can probably be explained in the following manner. When the hybridization is carried out in a single step, the target nucleotide sequence dissociated into a single strand is present in the solution in the presence of its strand of complementary target nucleotide sequence but also of the detection nucleotide sequence (and possibly the primers) and on the support for the trapping nucleotide sequence. The target nucleotide sequence can either first react with the detection nucleotide sequence before attaching to the trapping agent, or can attach to the trapping agent before attaching the detection nucleotide sequence. In the 2 cases, the use of large nucleotide sequences will promote the speed of reaction as well as the stability of the hybrids formed. However, in this intermediate state, where only the nucleotide sequence is hybridized, the target nucleotide sequence possesses a portion of its unpaired sequence which can then be recognised by a complementary target nucleotide sequence which can redisplace the intermediate hybrid and reform a double-stranded target nucleotide sequence. Consequently, when the target nucleotide sequence will be hybridized and completely overlapped by the trapping nucleotide sequence and by the detection nucleotide sequence without having any (or too few) free sequences, the annealing of another strand of the complementary target nucleotide sequence can no longer take place and the sandwich hybrid will be stable. In the case where the target nucleotide sequence even after sandwich hybridization retains a free sequence, the latter can still serve as annealing site for the other strand of the complementary target nucleotide sequence, which will destabilize the hybrid and can even cause it to dissociate depending on the experimental conditions (temperature, salts and the like), because once the annealing has started, the propagation of the formation of the double strand is very rapid and thermodynamically favorable.

This optimization of the various parameters and compounds involved in this sandwich hybridization makes it possible to carry out this sandwich hybridization both with a labeled nucleotide sequence present in the solution and a trapping nucleotide sequence attached to an insoluble support at the same time whereas if this is not the case, a hybridization should first be carried out in solution before carrying out the capture on an immobilized nucleotide sequence as described by Ghost et al. (EP 557456) or using a more complex sandwich hybridization system in solution followed by capture with a receptor attached to a solid support (Miller, U.S. Pat. No. 5,374,524).

Another characteristic of the sandwich hybridization method according to the invention is that not only can the target sequence completely overlap the trapping nucleotide sequence and the labeled nucleotide sequence, but that the trapping sequence can completely overlap with the target sequence. This therefore makes it possible to use, as labeled nucleotide sequence, large double-stranded nucleotide sequences easily produced for example by PCR amplification in the presence of dUTP-biotin. Indeed, once one of these biotinylated strands has annealed with the target nucleotide sequence, it will be completely overlapped and will no longer be able to be redisplaced by its complementary strand, which is not the case in this work reported by Keller et al. (1989, Anal. Bioch. 177, 27-32). This invention therefore allows easy production, in a very large quantity, on the one hand, of trapping nucleotide sequences using a primer carrying a 5′ terminal phosphate which will allow the covalent attachment of only one of these strands onto the aminated microplates and, on the other hand, of the labeled nucleotide sequences. If the labeled sequences are small, from 20-30 or 40 bases, they will be chemically synthesized and will be single-stranded. If they are larger, they can be produced by amplification.

The extension of the target nucleotide sequence outside the portion hybridized to the capturing agent and the detection nucleotide sequence via its 5′ terminal end caused a decrease in the hybridization yield if this noncovered portion became large. This is also the case if the 3′ terminal portion is not covered. By choosing optimum reaction conditions, a target nucleotide sequence of 20 bases binds to a trapping nucleotide sequence of 20 bases which is complementary to it with a yield which may be 25 times higher relative to a sequence which has the same 20 nucleotides but which has, in addition, 20 other additional nucleotides on the 3′ terminal side. A 25-fold difference in efficiency is therefore seen between these 2 sequences because of the presence of a piece of the nonhybridized sequence on the 3′ side. The explanation for such an effect is undoubtedly due to a steric hindrance effect of this free sequence situated near the support. This unexpected effect can be used in order to be able to measure small nucleotide sequences in the presence of larger sequences, this is the case in amplification methods such as CPR where the reaction product to be measured is a small sequence obtained from a larger starting nucleotide sequence.

The invention relates to the construction of one or more oligonucleotide sequences (DNA or RNA) having particular specificities, as described below, and their use as standards for the measurement of target DNA or RNA sequence(s) by sandwich hybridization with the aid of oligonucleotide probes labeled according to the method described above or otherwise.

The construct represented in FIG. 4 of this “standard” sequence 1 was designed so that, in the event of a possible prior amplification, the efficiency of the amplification is identical or very similar to that of the target sequence 2 to be quantified and so that the amplicons of the standard and of the target can be quantified with an equal or very similar efficiency.

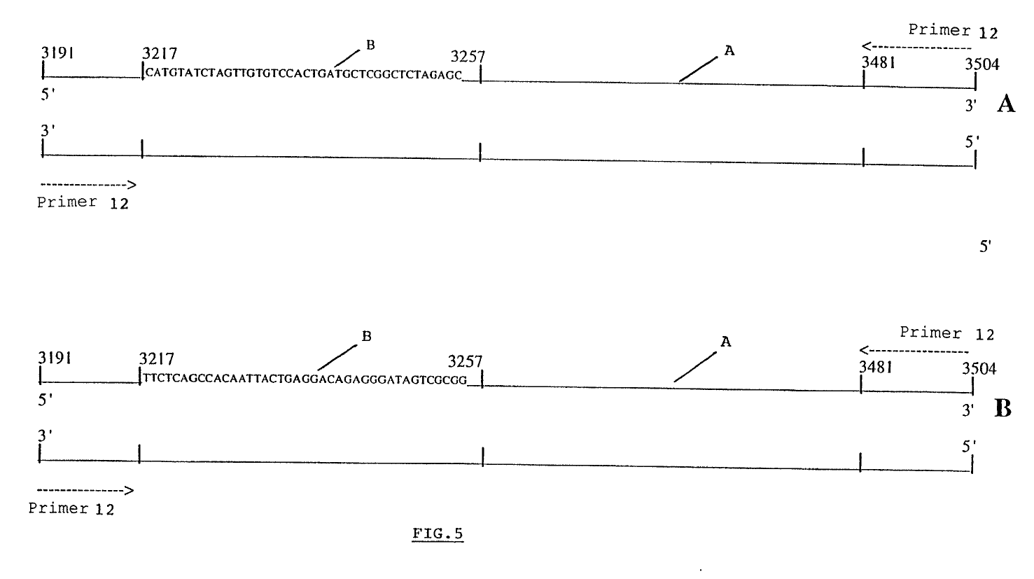

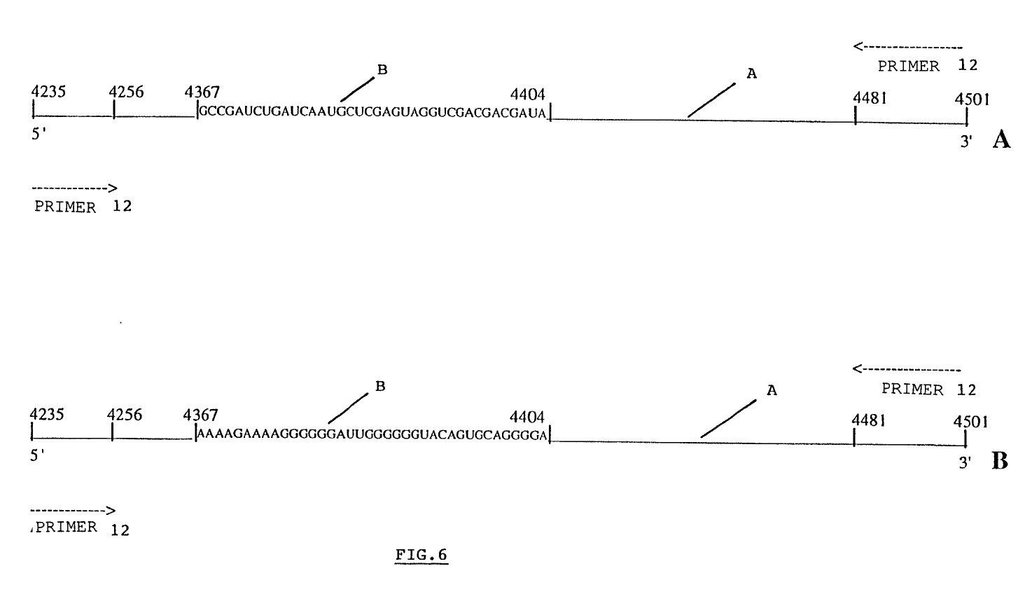

The standard 1 according to the invention is compatible with a possible prior amplification and a quantitative detection of a DNA (or RNA) sequence of which a portion A of the sequence is identical to the target DNA and at least a portion B (as small as possible) is different. In the case of a PCR amplification, these two portions will be flanked by two sequences 3 identical to those of the target DNA or RNA which will serve as template for the attachment of primer oligonucleotides (primers) 4. The length of the standard will be identical or very similar to that of the target DNA or RNA. Care will also be taken to introduce into the specific portion B a AT and GC base content, close to or identical to that of the target DNA or RNA. An example of a standard used for the quantification of a DNA fragment of the CMV virus is given in FIG. 5, and an example of an RNA standard for the quantification of an RNA sequence of the HIV virus is shown in FIG. 6.

Such a standard also allows use as an external standard or as an internal standard for the PCR amplification. Indeed, their similarity and partial identity allows an amplification level which is very similar or identical to that of the target DNA or RNA. They possess template sequences 3 for the identical primers 4, which will therefore achieve their annealing in the same proportions. Their identical length, their sequence identity over a long distance and the similarity of the noncommon portion in terms of length and in terms of ratio number of GC bases/number of AT bases is such that the reading by DNA polymerase will be carried out with the same efficiency. In the case of a use as internal standard, it also possesses a particular property. Indeed, in the amplification phase, the slowing down and the termination of the amplification of the standard sequence causes the same slowing down for the target sequence and conversely. This slowing down of the efficiency at the end of the amplification process may be due to several reasons: the reduction in the number of primers, the number of free nucleotides, the decrease in activity of DNA polymerase or the too rapid rehybridization of the amplicons with each other rather than with the primers during the primer annealing step. All these reasons are equivalent for the 2 sequences and will therefore have the same influence on their amplification. The latter property is advantageous because when one of the two amplicons (for example the standard) will be in a high concentration, it will recombine into a double strand in preference to the attachment to the smallest and therefore less stable primers. However, since it has a large sequence in common with the other amplicon (for example the target), it will also re-form stable recombinations (hybrids) with this other amplicon, which will inhibit in the same manner the attachment of the primers. Thus, the design of the competitive standards according to the invention allows quantification independently of the PCR cycles. An example is given in FIG. 15 showing the possibility of quantification after 25, 30, 35 and 40 cycles, a point where the PCR amplification is no longer exponential.

Conjointly to their use for the amplification, the presence on these standards of a common portion A and of a specific portion B is advantageously suited to their measurement according to the “sandwich hybridization” method on an insoluble support of the invention, and this with a specificity identical or very similar to that of the target nucleic acid (see FIG. 4).

The inventors have observed that by the sandwich hybridization of the invention, the limiting factor is the attachment of the amplicons 1 and 2 to the immobilized trapping sequence 5. Given that the latter is present on a surface, the speed of reaction is much slower because of the slower diffusion of the reagents on approaching a rigid surface and the steric hindrances. Experimentally, it is observed that less than 50% of the amplicons 1 and 2 are attached to the trapping sequences 5. However, even if this percentage attachment is low, it is observed that it is the same for the standard 1 and for the target 2. This property can be explained by the fact that the 2 sequences have the same size and that they attach to a trapping sequence 5 of the same type. The attachment of the labeled specific sequences 6 does not appear to pose any problem because a single-stranded probe is involved, which is introduced in excess so as to obtain a quantitative attachment. Experimentally, it is possible to obtain 90% attachment of these labeled sequences 6 to the amplicons 1 and 2 trapped in the wells. Furthermore, the identical length of these sequences 6 and their content which is comparable to a greater or lesser degree in GC and the identical concentration used cause the 2 specific sequences 6 complementary to the standard 1 and the target 2 to bind with the same efficiency. Thus, the entire sandwich hybridization is carried out with the same efficiency for the standard amplicons 1 and for the targets 2. This is illustrated experimentally in FIG. 8.

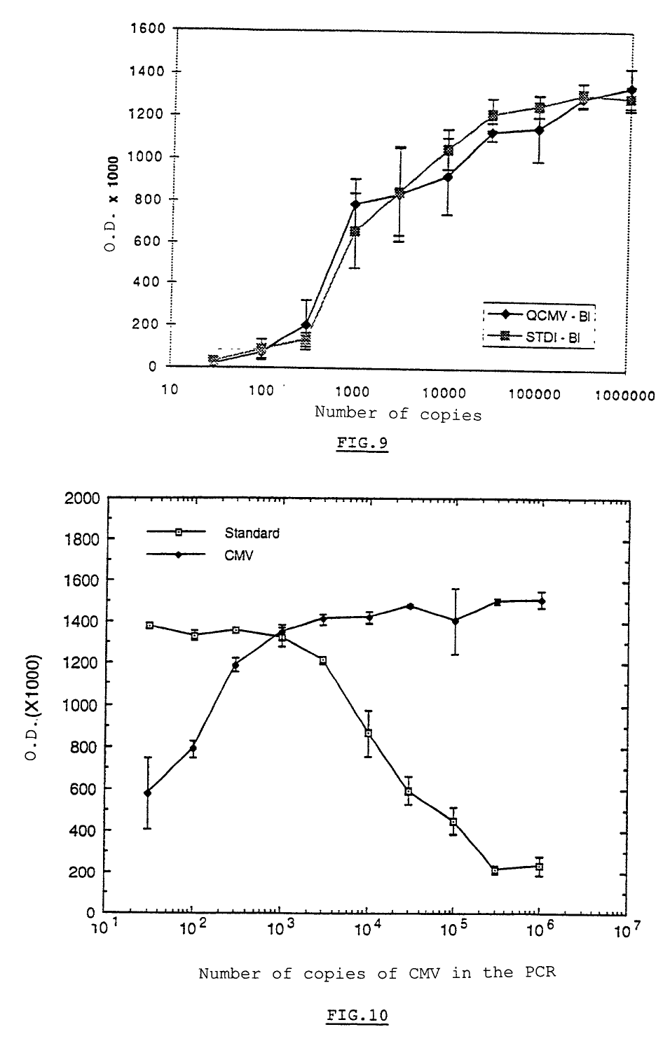

It is therefore advantageously possible to use these standards as external standards since their amplification efficiency will be identical and also their measurement by sandwich hybridization. An experiment of this type is shown in FIG. 9 in which increasing numbers of copies of target and standard CMV were amplified by 40 PCR cycles and then detected by sandwich hybridization. A parallel variation of the two curves is observed. A certain variability in the results resulting essentially from the variabilities of the amplification of the same sample during the PCR as explicitly stated above should however be underlined.

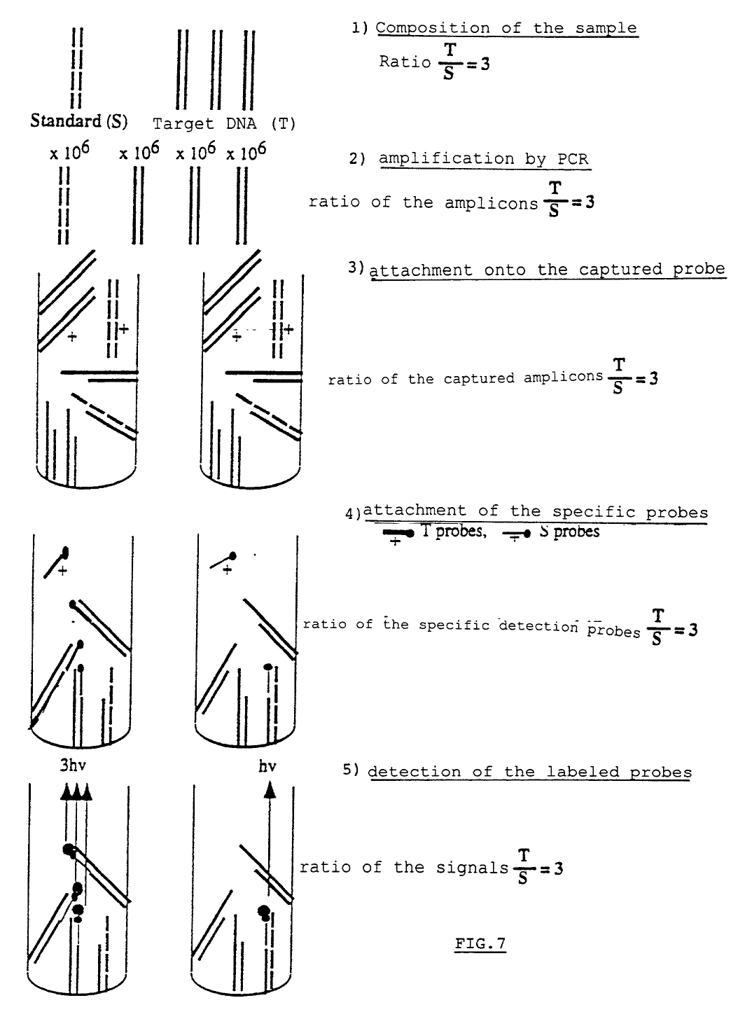

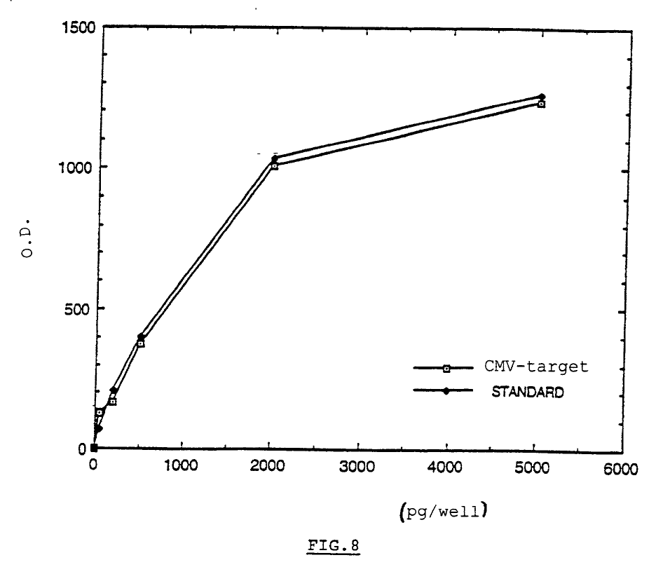

In the case where the standard is used as internal standard (that is to say that after the amplification by PCR, the tubes contain a mixture of standard and target amplicons) this sandwich hybridization also allows quantification of these two amplicons with the same efficiency operating in the following manner: the preparation containing the 2 amplicons is suitably diluted and is added to 2 or a double series of wells (or filters or tubes) in which a trapping DNA corresponding to all or part of the sequence common to the two amplicons is present. To one well (or a series of wells), a labeled sequence specific to the target is added and to the other well (or 2nd series of wells), the labeled sequence specific to the standard is added (see FIG. 7).

For a given dilution, the attachment of the two amplicons to the trapping DNA will be identical in the 2 tubes since they will be present at the same concentration. Furthermore, since the trapping agent is common to the two amplicons and since they have an identical size, their attachment will take place with the same yield, that is to say that the proportion of the two amplicons attached will be identical to that present in the preparation after PCR. Their relative concentration at the bottoms of the wells will therefore depend only on their respective concentration in the sample. If the labeled specific sequences are added in excess such that their attachment to the amplicons is quantitative, a labeling will be obtained which will be directly proportional to the concentration of the amplicons in the preparation.

The sandwich hybridization can be carried out either in two steps or in one step, by carrying out the attachment to the trapping sequence and then by adding the specific labeled sequence or by adding together the specific labeled sequence to the amplicons during the immobilization to the trapping sequences.

The use of standards according to the invention therefore makes it possible to obtain not only an identical (or very similar) amplification of the standard and of the target DNA but also their capture and their detection in an identical proportion in the tubes which serve for measuring the target amplicons and the standards. The measurement of the labeled sequences should be carried out subsequently in a quantitative manner so as to retain the quantification at all steps. A diagram representing the various steps of the quantification procedure with the aid of internal standards is presented in FIG. 7.

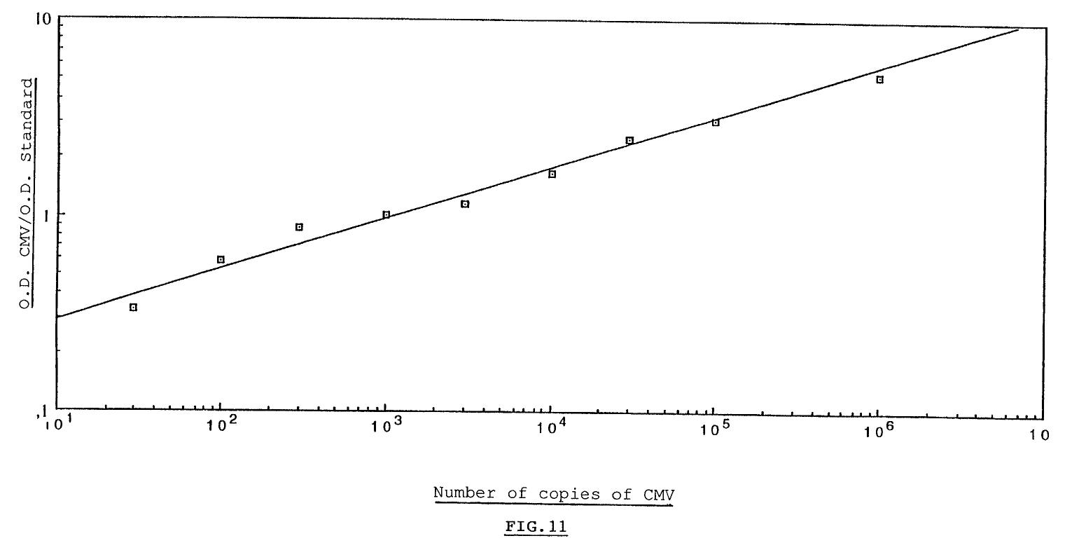

Beside the particular properties during the amplification by PCR resulting from the design of the internal standard which are cited above, namely an efficiency identical to that of the target in the biological sample or an independence relative to the number of PCR cycles, the quantification by sandwich hybridization as proposed in this patent and carried out according to a protocol represented in FIG. 7 provides a further advantage which can be observed in Example 9 (of which the results are presented in FIGS. 10 and 11). In this experiment, a constant quantity of internal standard, that is to say a thousand copies, were added to increasing quantities of CMV targets ranging from 30 to one million copies. After 40 amplification cycles by PCR, the 2 sequences were measured after sandwich hybridization by spectrophotometric measurement. FIG. 10 represents the data for the standards and the targets. A substantial presence of the standard is observed at low concentrations of target which decreases as the quantity of targets increases. When the quantity of targets is equal to that of the standard, that is to say one thousand copies, the two values are practically identical. Since one of the properties of the invention is to be able to retain the constant ratios between the standard and the target at each of the steps of the quantification, the ratios of the signals obtained for the measurement of the target and standard nucleotide sequence were plotted as a function of the quantity of target sequences introduced at the beginning. A linear relationship is effectively obtained, which indeed confirms the relevance of the results of the invention. An unexpected property was to observe this linearity on such a large scale of target nucleotide sequences. Indeed, the results indicate the possibility of quantitatively detecting between 30 and one million copies of these target sequences using at the beginning a thousand copies of standard sequences. This property constitutes an important practical advantage because the biological samples may contain highly variable quantities of target nucleotide sequences to be measured and in this case, a linear assay or more than 4 orders of magnitude makes it possible to substantially, if not completely, reduce the number of dilutions to be carried out in order to be in the quantification region.

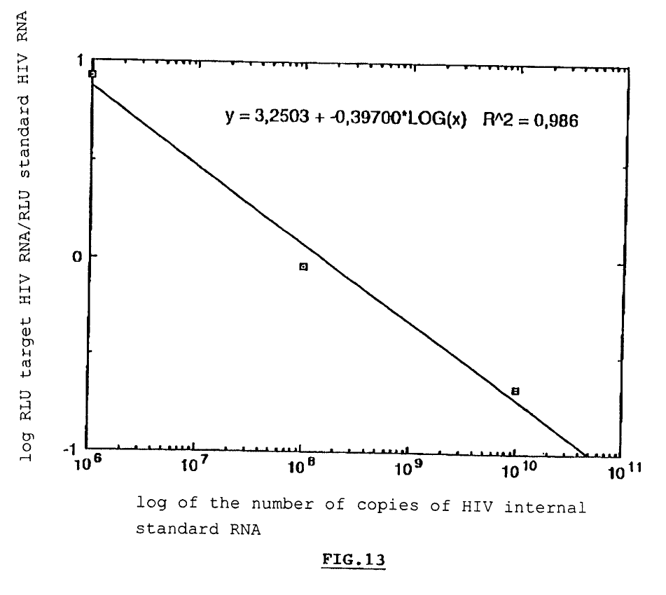

The same approach may be carried out during the measurement of RNA, for example messenger RNAs or viral RNAs. In this case, it is necessary to use a standard which consists of an RNA chain having the same specificities as the DNA standard explicitly described above. The approach for the amplification and the measurement requires a preliminary step which is the conversion of the RNA to a DNA chain by an enzyme having a reverse transcriptase activity. The remainder of the operations and the quantification is identical to that explicitly stated above. An example of the standard used for the quantification of HIV virus RNA is given in FIG. 6 and the quantification of HIV presented in Example 11 and in FIG. 13.

The use of these standards therefore allows the quantification of a target DNA (or RNA) sequence in a sample and is therefore very suitable for tests for screening or measuring DNA or RNA in research or in routine tests. The use of oligonucleotides as external standards is less advantageous than their use as internal standards and it requires special conditions and controls. It is necessary to be certain that the efficiency of the amplification is identical for the sample and for the tube containing the external standard; it is also necessary to work in a linear amplification region in order to maintain proportionality between the number of amplicons obtained and the quantity of sequences present in the sample and the standard at the beginning. It will also be necessary to carry out several replicate tests in order to minimize the variations observed from one tube to another during the amplification. On the other hand, the advantages given by the specific composition of this standard which is very similar to the target nucleic acid sequence are maintained in order to obtain an amplification and a detection identical or very similar to that for the target nucleic acid, which allows the quantification of the latter.

In practice, the sample containing the target DNA or RNA is treated in parallel with tubes containing increasing concentrations of external standard. All the amplification and detection conditions are identical and are produced with the same solutions in order to minimize the variations in treatment. The results are compared with the calibration curve obtained with the standards. In the invention, a series of dilutions of the target sample is preferred in order to obtain values corresponding to the region covered by the standard.

The use of the internal standards not only makes it possible to take into account variations which occur from one tube to another during the PCR, but also possible inhibitions of the PCR which may occur in biological samples. These are in general due to an inhibition of the activity of polymerase by contaminants present in the preparation. In the case of inhibition of the amplification, the latter will be produced on the 2 sequences, the target and the standard, which are present in the same tube and the proportion of the amplicons will therefore be maintained in spite of a lower amplification yield. Furthermore, this inhibition of the PCR may be observed and evaluated by comparing the measurement of the amplicons of the standard added to the sample and that of the positive control for the PCR. In the case of inhibition, the signal of the internal standard present in the highly diluted sample will be less than that of the control containing the same number of copies of the internal standard treated under the optimum PCR conditions.

In practice, a known quantity of standard is added to the sample containing the target DNA or RNA and then the preparation for the amplification and detection is treated after sandwich hybridization. A positive control containing the standard alone and a negative control with no specific DNA are also produced for each experiment. The ratio of the signal obtained for the measurement of the target amplicons and the amplicons of the standard is compared with a calibration straight line (see FIG. 11) which makes it possible to determine the number of copies of target DNA at the beginning.

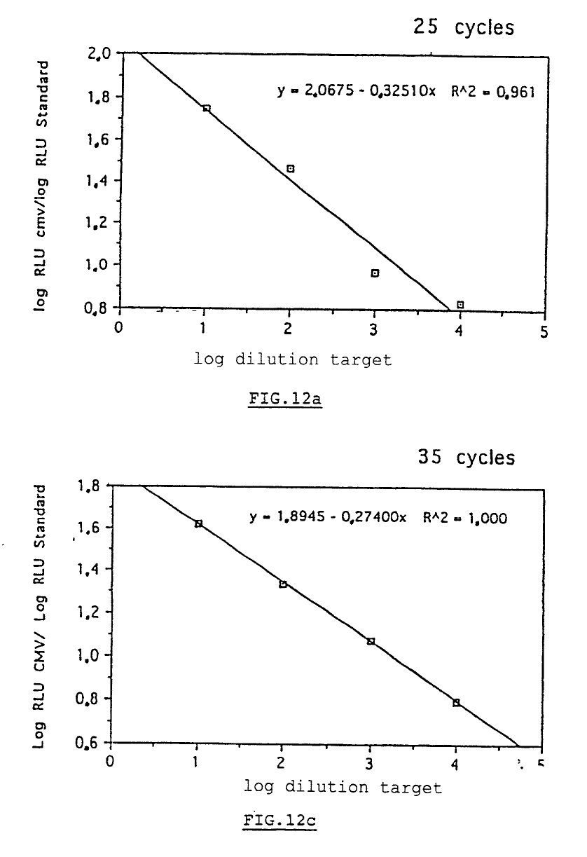

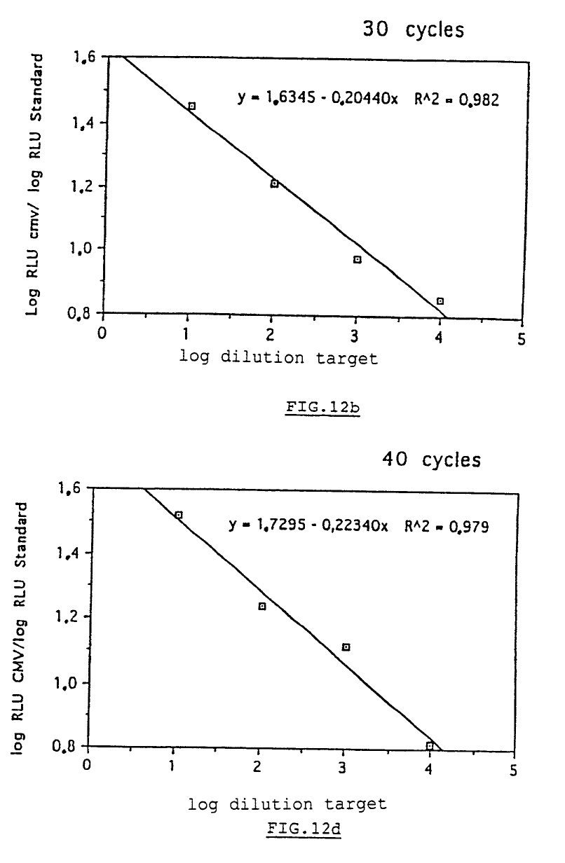

A method which is more suitable but which requires more tests consists in diluting the sample (for example 4 10-fold dilutions) and to add thereto a constant quantity of internal standard. For certain biological samples, it is necessary to extract and even sometimes purify the nucleic acids before the amplification in order to avoid inactivation of the polymerase. In others, the heating to 100° C. intended to separate the double strands of DNA is sufficient. The internal standard is normally added to the starting sample. It may optionally, for practical reasons, be added after extraction if the latter is quantitative. This is the case where the nucleic acids are extracted from a sample and then 4 dilutions are carried out before adding a constant quantity of internal standards before carrying out the quantification as in Example 10 and FIG. 12. This manner of proceeding only requires a single extraction of the sample. After amplification and detection after sandwich hybridization, the ratios between the signals of the target and standard amplicons are plotted as a function of the dilution of the target. These 4 points determine a straight line which makes it possible to determine the value of the ratio equal to 1 for which the quantity of target is equal to that of the standard. This manner of carrying out the quantification is optimal. An example is explicitly given below and presented in FIG. 12.

### EXAMPLE 1

Influence of the length of the trapping agent for a simple hybridization of a HPV-18 target DNA

The experiment was carried out on amplicons of 586 base pairs corresponding to a HPV-18 sequence situated at position 6193 to 6779 of the viral DNA. This sequence was amplified by the following primers:

1=5′ TTTTGGAAGATGGTGATATGG 3′

2=5′ CATAACATCTGCAGTTAAAGT 3′

The hybridization was carried out using these amplicons labeled at the 5′ terminal end by means of T4 polynucleotide kinase in the presence of [γATP with ³²P].

Two types of trapping agents were used, corresponding to 180 and 360 bases complementary to the sequence of the amplicons. The hybridizations were carried out in the presence of increasing concentrations of target amplicons at 45° C. for 20 hours in a solution consisting of 2× concentrated SSC, 5× concentrated Denhart, denatured salmon sperm DNA at 0.1 mg/ml.

Concentrations of the amplicons ranging from 0.13 to 13 fmol were tested, and the quantity attached was measured by radioactivity. Expressed as a percentage of bound DNA, this represents 30% on the trapping agent of 180 bases and 50% on the trapping agent of 360 bases regardless of the target concentrations tested.

The fact that the percentage is stable between 0.13 and 13 fmol also indicates that the quantity of trapping agent is not limiting.

### EXAMPLE 2

Effect of the length of the trapping agent on a simple hybridization of a CMV target DNA

The experiment was carried out for a hybridization of amplicons of 435 base pairs corresponding to a CMV sequence at position 171075 of the genome (AD169 strain, reference GENBANK X17403).

This sequence was amplified by the following primers:

MIE4: sense: 5′ CCAAGCGGCCTCTGATAACCAAG

MIE5: antisense: 5′ CAGCACCATCCTCCTCTTCCTCTGG

as described by Demmler et al. (J. Infect. Dis., 158, pp: 1177-1184 (1988)) and labeled with ³²P using during the amplification by PCR dCTPs labeled with ³²P. The trapping agents of 50, 100, 150 and 250 bases were produced by PCR and correspond to the 3′ terminal portion of the amplicons.

The hybridization was carried out at 60° C. for 2 hours in a solution of 2× concentrated SSC and 5× concentrated Denhart in the presence of 0.1 mg/ml of salmon sperm DNA. A quantity of 10 fmol of ³²P amplicons was added to each well. After reaction, the wells were uncoupled and the radioactivity measured. The results show that for the trapping agents of 50, 100, 150 and 250 bases, 0.09; 0.35; 0.53 and 1.55 fmol of hybridized target sequence are obtained respectively. A spectacular effect of the length of the trapping agent on the hybridization is indeed observed.

### EXAMPLE 3

Influence of the length of the trapping agent on the sandwich hybridization of an HPV-18 target DNA

The experiment was carried out for the hybridization of amplicons of 586 base pairs corresponding to an HPV-18 sequence situated at position 6193 to 6779 of the viral DNA. This sequence was amplified by the primers described in Example 2.

Three types of trapping agents were used, corresponding to a sequence of 25, 180 and 360 bases complementary to the sequence of the amplicons.

The various trapping agents recognise the portion of the amplicons situated on the 3′ terminal side. The probes comprised either 360 bases for the hybridization on the trapping agent of 180 bases, or 180 bases for the hybridization on the trapping agent of 360 bases, or 21 bases for the hybridization on the trapping agent of 25 bases. They were labeled with ³²P by phosphorylation at the 5′ terminal position by T4 kinase in the presence of [³²P]ATP. They corresponded to the 5′ terminal end of the target sequence. The probes of 21 bases were single stranded, whereas the probes of 180 and 360 bases were double stranded. The sandwich hybridizations were optimized as regards the temperature, the concentration of salts and the concentration of amplicons. As regards the trapping agent of 25 bases, the hybridization carried out at 45° C. in the presence of 2.5 fmol of amplicons for 2 hours in a solution of 2× concentrated SSC and then after washing incubated for 2 hours with 15 fmol of probe labeled with ³²P. An attachment of 0.280 fmol is obtained with a coefficient of variation of 80%. The hybridization on the trapping agents of 180 and 360 bases occurred in a single step and under the following conditions: the amplicons were added respectively in an amount of 2.5 fmol in the presence of 15 fmol of labeled probe. The hybridization occurred for 20 hours in a 2× concentrated SSC solution at 45° C. The quantity of labeled probe hybridized is 0.54 fmol for the trapping agent of 180 bases and 0.69 fmol for the trapping agent of 360 bases. The difference in attachment of the probe was due to a higher yield of hybridization of a target sequence onto the large trapping agent (cf. Example 2).

Indeed, if the attachment of the labeled probe is expressed in terms of the quantity of trapped target sequence, a ratio of 0.8 is obtained in both cases. This means that 80% of the target sequences hybridized to the capture sequence attached the labeled sequences. It is therefore observed in this example that it is possible to use labeled detection probes which are even double stranded and to obtain a very good yield of their hybridization (80%). The limiting factor for the formation of the sandwich under these conditions is then the length of the capture probe.

### EXAMPLE 4

Influence of the overlapping of the target nucleotide sequence with the trapping DNA and the probe DNA for the hybridization yield (example of Mycobacterium tuberculosis)

In this example, the DNA extracted from Mycobacterium tuberculosis was amplified by its Mt 308 sequence using the following primers:

T2MT3′ (primer 5′-3′):

GTCGACACGCCTCTGCACGGAAGTCCTT

DMT3′ (antisense primer 5′-3′):

GCTCGACTTCTGGTCACGACGTCCGTCGAA

These primers were used by Thierry et al. (Mol. Cell. Probes, 6, p. 181 (1992)), and allow the amplification of a fragment of 279 base pairs.

The trapping agent was obtained using the probe T2MT3′ phosphorylated at the 5′ position and an antisense primer SMT5′: GGGCATCCGCGAGTTGAAGACCTGAAGTGG.

These two primers allow the production of amplicons of 144 base pairs, in which one of the two strands has a phosphate in 5′. The latter was used to attach the trapping agent by a covalent strand onto the amine of the multiwells as explained in Example 1.

Two labeled probes were produced, one of 135 base pairs obtained by the use of an antisense primer GSMT540 carrying a biotin:

TCATTGGCAACGTTTGCGCCCTGCCTTGGG and the other by the antisense primer DMT3′. The other probe was single stranded and also carried a biotin:

5′ CAGCCACCAAGTCGAGCACTTTGCGGCGGAACTACTCGGG-biotin 3′

The sandwich hybridization was carried out by adding to each well 54 fmol of target DNA amplified by PCR in the presence of 50 ng of probe of 40 bases and of 25 ng for the sequence of 135 bases. The hybridization was carried out for 2 hours at 60° C.

After washing, the streptavidin-kinase conjugate was added and after 45 minutes of incubation, the activity of the kinase was assayed by bioluminescence as described in Patent Application WO94/06933.

The reading is made for one hour. After subtracting the blank, a value of 1.5 million RLU (Relative Light Unit) is obtained for the probe of 40 bases and 2.7 million RLU for the probe of 135 bases.

In this example, the probe of 135 bases was double stranded and used in a smaller quantity. In spite of these two unfavorable conditions, the signal obtained is almost twice greater, which indeed indicates the importance of producing a large detection probe which covers the entire target sequence.

### EXAMPLE 5

Curve of concentrations of CMV amplicons after sandwich hybridization

The amplicons obtained from CMV DNA were obtained from a PCR carried out with the primers MIE4 and MIME5 described in Example 2. They make it possible to obtain amplicons of 435 base pairs. The amplicons were used to produce the sandwich hybridization curve described below. This was produced on multiwell plates on which were attached trapping agents complementary to this target sequence and a biotinylated probe of 185 bases also produced by PCR. The size of the trapping agent was 257 bases. They were produced from a PCR using primers MIE4 described in Example 3 and MEI-6 whose sequence was:

GTACAGGGGACTCTGGGGGTGAC

The trapping agent once produced is purified on a G25 spin column.

The covalent attachment of the trapping agent onto the polystyrene is done as follows: each well containing 100 ng of denatured trapping agent, 0.01 M MIEM pH 7.5, 0.2 M carbodiimide. These wells are incubated for 5 hours at 50° C. After incubation, two washes with 200 μl of 0.2 N NaOH, washing with water and drying are carried out. For the hybridization, the wells are denatured with 200 μl of 0.2 N NaOH and rinsed with 200 μl of 2× SSC.

The total hybridization volume per well is 110 μl, containing:

50 μl of hybridization buffer: 4.4× SSC, 10× Denhart, salmon sperm DNA 200 μg/ml denatured 10 minutes at 100° C.;

10 μl of biotinylated probe at a concentration of 900 pg/10 μl denatured 10 minutes at 100° C.;

50 μl of target DNA amplified by PCR using two primers MIE4 and MIE5 described in Example 3, not purified and denatured 10 minutes at 100° C. The quantities tested range from 3.5 attomoles to 3500 attomoles.

The hybridization lasts for 2 hours at 70° C.

After hybridization, the wells are washed twice with 200 μl of 0.1× SSC and then with 200 μl of 100 mM maleate buffer, 150 mM NaCl, 0.3% Tween pH 7.5 for 15 minutes.

After rinsing with 200 μl of 100 mM maleate buffer pH 7.5 containing 150 mM NaCl and 1% blocking reagent, 100 μl of streptavidin-kinase are added. The wells are rinsed with 400 μl of 100 mM maleate buffer pH 7.5 containing 150 mM NaCl, 0.3% Tween for 10 minutes and then 3 times 5 minutes with 200 μl of 100 mM maleate buffer, 150 mM NaCl, 0.3% Tween pH 7.5.

The activity of the kinase is revealed in 20 mM Tris buffer pH 7.75 containing 60 μM DTT, 100 μM EDTA, 5 mM MgCl₂, 8 μM Luciferin, 6 mU luciferase per well 20 mM KCl, 1 mM phosphoenolpyruvate and 3.2 μM ADP. The emission of light is monitored for one hour with a luminometer (Luminoskan, Labsystem, Finland) and the results are expressed in RLU.min.

### EXAMPLE 6

Differential hybridization of a short and of a long DNA strand having a portion of its sequence which is identical

During some amplifications such as CPR, the labeled starting probes are large and are cut into two pieces which should be able to be detected and measured. The problem is therefore to be able to measure, by hybridization, a small probe in the presence of its mother sequence which is larger. The inventors chose in this example a biotinylated mother sequence (OL1) of 40 bases which have the following sequence:

5′ CCGCGACTATCCCTCTGTCCTCAGTAATTGTGGCTGAGAA 3′

This sequence corresponds to a specific sequence of the CMV genome situated on the “Major Immediate Early Gene” (Akrigg et al., Virus Res. 2, p. 107). The inventors wanted to detect a biotinylated probe (OL2) corresponding to the first 20 bases of this probe in the presence of the mother sequence (OL1). The trapping agent consisted of an oligonucleotide of 20 bases complementary to the probe OL2 and ending with a phosphate group at 5′. This trapping agent was attached to the aminated multiwells by a covalent reaction.

In the following experiment, 100 fmol of two biotinylated sequences OL1 or OL2 were incubated for 2 hours at 45° C. in a 0.5× concentrated SSC solution (that is to say 75 mM NaCl and 7.5 mM Na nitrate) . After hybridization, the wells were washed with a 0.1× concentrated SSC solution at 45° C. After four washes, the wells are incubated with the streptavidin-kinase conjugate and its attachment is estimated by the measurement of the kinase activity by bioluminescence. Under these conditions, the wells having the large fragment (OLA) showed an RLU×min of 9 whereas the small fragments showed an RLU×min of 210. The presence on OL1 of a sequence of 20 additional nucleotides on OL1 which are situated on the 5′ side relative to OL2, that is to say on the side of the plastic, therefore greatly destabilizes the hybridization of this probe onto the trapping agent.

### EXAMPLE 7

Measurement of the quantity of target DNA and of standard by sandwich hybridization and measured by spectrophotometry

The target DNA to be measured consists of a double-stranded oligonucleotide of 314 base pairs corresponding to position 171193 of the CMV genome (strain 10169, reference GENBANK X17403).

The internal standard consists of a double-stranded oligonucleotide of 314 base pairs whose nucleotide sequence is identical to that of the target DNA to be measured except for the 40 nucleotides ranging from 3217 to 3256 which constitute a random sequence but whose percentage of GC is similar to that of the target DNA.

The composition of these nucleotides is presented in FIG. 5. These nucleotides are first heated at 100° C. for 10 min and then incubated in an increasing concentration in wells to which there have been attached capture probes of the invention and corresponding to a portion of the common sequence and provided by Lambdatech (Namur-Belgium). The hybridization solution of 0.06 ml per well contains a twice concentrated ssC solution, 5 times concentrated Denhardt, 100 μg/ml of denatured salmon sperm DNA and 50 ng of biotinylated probe of 40 single-stranded nucleotides corresponding either to the sequence complementary to the target DNA, or to that of the standard DNA presented in FIG. 7. 40 μl of target or standard DNA are added to these 60 μl. The hybridization is carried out at 70° C. for 2 hours.

After hybridization, the wells are washed once with 0.2 ml of 0.1 times SSC solution, and then once with 0.2 ml of maleate buffer pH 7.5 containing 0.15 M NaCl and 0.3% Tween and finally with maleate buffer pH 7.5, 0.15 M NaCl and containing 1% milk powder.

A streptavidin-peroxidase conjugate is added in 0.1 ml at a 1/1000 dilution as recommended by the supplier (BIOSOURCE-Fleurus-Belgium) and then washed 3 times with 0.2 ml of 0.1 M maleate buffer pH 7.5 containing 0.15 M NaCl and 0.3% Tween and then once with a 500 mM solution of glycine pH 7.7 containing 100 mM KCl and 1 M MgCl₂.

The peroxidase activity is measured by oxidation of TMB in the presence of H₂O₂ in a 1.1 ml of 0.2 M acetate-citrate buffer pH 7.5.

After 10 min of reaction, 0.22 ml of a 1.2 M solution of H₂SO₄ is added to each well and the optical density is measured at 450 mM. The results of the example are presented in FIG. 8.

### EXAMPLE 8

Measurement of the quantity of CMV target DNA using an external standard by sandwich hybridization after PCR amplification

CMV virus DNA contained in a plasmid whose copy number was known was placed in increasing concentrations in the PCR tubes. In parallel, tubes containing increasing concentrations of external standard as defined in FIG. 5 are prepared. All the tubes were subjected to a PCR of 40 cycles using primers corresponding to the sequence 3191-3217 and 3504-3481 of the CMV gene (FIG. 5). The PCR starts with 1 passage 3 min at 94° C. and continues with 40 cycles defined as follows:

Each PCR cycle comprised a denaturing temperature at 94° C. for 30 sec, a primer annealing period at 65° C. for 30 sec and a polymerization of 30 sec at 72° C. The 0.1 ml PCR solution comprised 100 pmol of each of the 2 primers, 200 mM of the various dNTPs and 2.5 U of Taq DNA polymerase in a 10 mM TRIS-HCl buffer pH 8.4 with 1.5 mM MgCl₂ and 50 mM KCl, 2% DMSO. At the end of the 40 cycles, the PCR tubes remain for 10 min at 72° C.

After amplification, 0.04 ml of the solution was collected in order to carry out the sandwich hybridization on the trapping probe attached to the multiwells which is common to the two amplicons. The presence of target amplicons and of standard amplicons was demonstrated using a biotinylated probe of 40 bases which is specific for each of the 2 sequences (FIG. 5).

The procedure is performed using a streptavidin-kinase conjugate and by measuring the activity of the kinase bioluminescence. The experimental conditions for the hybridization and the revealing are those of Example 7 but using a streptavidin-kinase conjugate which allows the production of light in the presence of luciferase as described in Patent Application WO94/06933. The results obtained are presented in FIG. 9. Each point represents the mean of 3 measurements. A correlation is observed between the starting number of copies and the signal obtained. Furthermore, the curves obtained for the target and the standard are very similar, which makes it possible to use the curve for the standard as reference to determine the number of copies of the CMV target DNA in the starting sample.

### EXAMPLE 9

Measurement of the quantity of CMV target DNA using an internal standard by sandwich hybridization after PCR amplification

In order to evaluate the method of measuring a target DNA in a sample using an internal standard and a PCR amplification as schematically represented in FIG. 7, a calibration curve is established in the following manner.

Increasing quantities of a plasmid containing a portion of the CMV virus genome and a constant quantity of internal standard, that is to say equivalent to 1000 copies, were added to each of the tubes. The quantities of CMV target DNA corresponded to 30, 100, 300, 1000, 3000, 10000, 30000, 100000, 300000 and 1 million copies.

The composition of the internal standard is given in FIG. 5. Each tube was subjected to an amplification using 40 cycles of PCR under the conditions presented in Example 15 using the primers corresponding to the sequence 3191-3217 and 3504-3481 of the CMV gene (FIG. 5). After amplification, amplicons of 314 base pairs corresponding to the CMV target and to the standard are therefore obtained. From each of the PCR solutions, 0.04 ml are removed and incubated in 2 wells containing an identical trapping agent for the common sequences of the 2 amplicons. The biotinylated probe of 40 bases which is specific for the CMV target is added to one of the wells and the biotinylated probe of 40 bases which is specific to the standard is added to the other.

After sandwich hybridization, a streptavidin-peroxidase conjugate is added to each tube and the peroxidase activity measured as in Example 7 by measuring the optical density (O.D.) corresponding to the absorbance of the product of the reaction of the peroxidase (FIG. 10). For each PCR, the ratio between the O.D. corresponding to the detection of the target amplicons and that corresponding to the detection of the standard amplicons is determined and plotted as a function of the number of CMV copies present at the beginning of the experiment. The results are presented in FIG. 11 on a logarithmic scale so as to cover all the concentrations. Each of the points represents the mean of 3 experiments. A very good linearity is observed with a regression coefficient of 0.97 for the straight line, which means the possibility of determining the quantity of target CMV in a starting sample ranging from 30 to 1 million copies.

### EXAMPLE 10

Independence of the number of PCR cycles for the measurement of a CMV target DNA using an internal standard and sandwich hybridization

This experiment was carried out essentially according to Example 9 and the diagram of FIG. 7, that is to say using at the beginning a sample containing 100000 copies of CMV DNA diluted 10-fold and to which 1 000 copies of standard were added. Each of these tubes was subjected to PCR as in Example 5 except that some tubes were stopped after 25, 30, 35 or 40 PCR cycles. After the PCR, the amplicons were used for a sandwich hybridization on the same trapping agent but with either a biotinylated probe specific for the target CMV or for the standard. The revealing is carried out by bioluminescence using a streptavidin-kinase conjugate and measurement of the light emitted by luciferase.

The ratio of the light signals (RLU) emitted by the wells using the probe for the target and those using the standard probe were plotted as a function of the dilution of the starting target (FIG. 12). For the same PCR, the linearity of the 4 points is observed, which means the possible detection in this case of the target CMV between 100 and 100000 copies. This linearity is found for the 4 PCRs comprising 25-30-35 or 40 cycles, which means the independence of this quantification relative to the number of PCR amplification cycles.

### EXAMPLE 11

Measurement of the quantity of target RNA and of standard by sandwich hybridization

The target RNA to be measured consists of a single-stranded nucleic acid of 267 bases corresponding to the nucleotide sequence ranging from 4235 to 4481 of the polymerase (Pol) gene of the Aids virus (HIV) (HIV-LAI numbering: Los Alamos HIV data bank ACK02013).

The standard consists of an RNA single-stranded nucleic acid whose nucleotide composition is identical to that of the target RNA to be measured with the exception of 38 nucleotides going from position 4367 to 4404, constituting a random sequence in which the percentage of GC is similar to that of the target RNA. The composition of these RNAs is presented in FIG. 8.

The RNA standard is prepared by in vitro transcription from a plasmid possessing in 5′ the promoter for an RNA polymerase and in 3′ of a poly A sequence. After purification and quantification of the latter, an increasing number of copies of RNA standard is placed in each tube in the presence of a fixed quantity of target RNA.

Each tube is subjected to a first reverse transcription step (AMV-RT, Avian Myeloblastosis Virus) for the synthesis of the first DNA strand (primer 4481-4501) and in a second step to the synthesis of the second cDNA strand as well as the amplification of the DNA (Tfl DNA polymerase Thermus flavius; Systm Access RT-PCR Promega) (primers 4235-4256; 4481-4501). An optimized reaction buffer for the two steps simplifies the procedure and reduces the risk of contamination.

The amplification procedure comprises, in a first step, reverse transcription at 48° C. for 60 min, followed by a step for inactivation of the AMV-RT and denaturation of the RNA/cDNA hybrids at 94° C. for 2 min. Next, the synthesis of the second cDNA strand and the PCR are carried out by an amplification of 40 cycles each comprising a denaturation at 94° C. for 30 sec, an annealing of the primers at 50° C. for 30 sec and an extension step at 72° C. for 30 sec. The RT-PCR reaction occurs in 50 μl of solution comprising as a final concentration 1 μM of each of the 2 primers, 0.2 mM of dNTPs, AMV/TFl reaction buffer, 1 mM MgSO₄, 0.1 u/μl AMV-RT and 0.1 u/μl Tfl DNA polymerase.

After amplification, the sandwich hybridization is carried out on the trapping probe common to the two amplicons which is attached to the multiwells. The presence of target and standard amplicons was demonstrated using a biotinylated probe of 38 bases which is specific for each of the 2 sequences. The revealing is carried out by bioluminescence using a streptavidin-kinase conjugate and measurement of the light emitted by luciferase (cf. Example 11).

The results of such an experiment in which 3 different concentrations of internal standards of 10¹⁰, 10⁸ and 10⁶ copies were added to a fixed concentration of 10⁸ copies of target HIV RNA are shown in FIG. 13.

1. Method for detecting and/or quantifying a target nucleotide sequence (2) present in a biological sample, characterized in that it comprises a bringing into contact of the “sandwich” type of the target nucleotide sequence (2) with a single-stranded trapping nucleotide sequence (5) attached to an insoluble solid support (3), and complementary to a portion (7) of the said target nucleotide sequence (2), and with one or more nucleotide sequences (6, 11) of which at least one is labeled (6), said nucleotide sequence(s) (6, 11) being complementary to another portion (8) of the target nucleotide sequence (2); in that the trapping nucleotide sequence (5) is covalently attached by one of its ends to the solid support (3); comprising a length of between 50 and 300 bases; and in that a portion (10) of the trapping nucleotide sequence (5) which does not hybridize with the portion (7) of the target nucleotide sequence (2) is less than 60 bases, preferably less than 40 bases, more particular less than 20 bases.

2. Method according to claim 1, characterized in that the length of the trapping nucleotide sequence (5) is between 120 and 250 bases.

3. Method according to claim 1 or 2, characterized in that the yield of hybridization of the target nucleotide sequence (2) to the trapping nucleotide sequence (5) is greater than 40%.

4. Method according to any one of the preceding claims, characterized in that a portion (13) of the target nucleotide sequence which does not hybridize with the trapping nucleotide sequence (5) and with the nucleotide sequence(s) labeled (6) or otherwise (11) is less than 60 bases, preferably less than 40 bases.

5. Method according to any one of the preceding claims, characterized in that the solid support (3) is a support chosen from the group consisting of tubes, filters, beads, which may be magnetic, multiwell plates or a mixture thereof.

6. Method according to any one of the preceding claims, characterized in that the target nucleotide sequence (2) is an amplicon resulting from a preliminary amplification by a gene amplification method, preferably chosen from the group consisting of PCR, LCR, CPR, NASBA or ICR.

7. Method according to claim 6, characterized in that the 5′ terminal portion (9) of the target sequence (2) not overlapping with the labeled nucleotide sequences (6) overlaps with a primer sequence (12) used for its amplification.

8. Method according to any one of the preceding claims, characterized in that it also comprises a bringing into contact of the sandwich type of a standard nucleotide sequence (1) under the same conditions as the target nucleotide sequences (2) to be detected and/or quantified and comprises an additional step of quantification of the ratio between the specific labeling of the target nucleotide sequence (2) and the specific labeling of the standard nucleotide sequence (1).

9. Method according to claim 8, characterized in that it also comprises a step of a preparation of a known quantity of a standard nucleotide sequence (1) possessing at least a portion (A) common to the target nucleotide sequence (2) and a specific portion (B) whose sequence is different and possesses a content of GC/AT bases similar, preferably identical, to the sequence of a specific portion (B) of the target nucleotide sequence (2), an optional extraction of the target nucleotide sequence (2) to be quantified from the biological sample and a bringing into contact of the “sandwich” type of the target (2) and standard (1) nucleotide sequences with trapping nucleotide sequences (5) complementary to the common portion (A) of these two sequences (1, 2) and with labeled nucleotide sequences (6) complementary either to the specific portion (B) of the target nucleotide sequence (2), or to the specific portion (B) of the standard nucleotide sequence (1), and a quantification between the specific labeling of the target nucleotide sequence (2) and the specific labeling of the standard nucleotide sequence (1).

10. Method according to any one of the preceding claims, characterized in that the sandwich hybridization is carried out in two steps.

11. Method according to any one of the preceding claims, characterized in that the attachment of the trapping nucleotide sequence (5) to the solid support (3) is carried out via a 5′ terminal phosphate onto an amine functional group of the solid support (3) by reaction with carbodiimide.

12. Method according to any one of the preceding claims, characterized in that the target nucleotide sequence (2) is a DNA.

13. Method according to any one of claims 1 to 11, characterized in that the target nucleotide sequence is an RNA.

14. Kit for detecting and/or quantifying, by a “sandwich”-type hybridization, a target nucleotide sequence (2) comprising a trapping nucleotide sequence (5) attached to an insoluble solid support (3) complementary to a portion (7) of the said target nucleotide sequence (2) and at least one labeled nucleotide sequence (6) complementary to another portion (8) of the target nucleotide sequence (2), characterized in that the said trapping nucleotide sequence (5) is covalently attached by one of its ends to the solid support (3) and has a length of between 50 and 300 bases, and in that the portion (10) of the trapping nucleotide sequence (5) which does not hybridize with a portion (7) complementary to the target nucleotide sequence (2) is less than 60 bases, preferably less than 40 bases, more particularly less than 20 bases.

15. Kit according to claim 14, characterized in that the length of the trapping nucleotide sequence (5) is between 120 and 250 bases.

16. Kit according to claim 14 or 15, characterized in that the solid support (3) is an insoluble solid support chosen from the group consisting of tubes, filters, beads, which may be magnetic, multiwell plates or a mixture thereof.

17. Kit according to any one of claims 14 to 16, characterized in that it comprises a standard nucleotide sequence (1).

18. Kit according to any one of claims 14 to 17, characterized in that the standard nucleotide sequence (1) possesses at least a portion (A) common to the target nucleotide sequence (2) and a specific portion (B) whose sequence is different and possesses a content of GC/AT bases similar, preferably identical, to the specific portion (B) of the target nucleotide sequence (2) to be quantified.

19. Kit according to any one of claims 14 to 18, characterized in that the trapping nucleotide sequence (5) is attached by a 5′ terminal phosphate onto an amine functional group of the solid support (3) by reaction with carbodiimide.
